(19) United States
(12) Patent Application Publication
(10) Pub. No.: US 2014/0079786 A1
(43) Pub. Date: Mar. 20, 2014

(54) Fiber Spheres

(71) Applicant: Grain Processing Corporation, Muscatine, IA (US)

(72) Inventors: Susan Freers, Muscatine, IA (US); Carrie Shipley, Davenport, IA (US); Brian Jensen, Cedar Rapids, IA (US); Shawn Engels, Tipton, IA (US)

(73) Assignee: Grain Processing Corporation, Muscatine, IA (US)

(21) Appl. No.: 14/031,781

(22) Filed: Sep. 19, 2013

Related U.S. Application Data
(60) Provisional application No. 61/703,053, filed on Sep. 19, 2012.

(51) Int. Cl. A61K 47/46; A23L 1/308

(52) U.S. Cl. CPC A61K 47/46; A23L 1/3081
USPC 424/490; 424/499; 514/570; 427/212; 426/615; 426/618; 426/289; 426/2

(57) ABSTRACT

The disclosure pertains generally to spheres made from a fiber source. These spheres may be used as cores for carrying other ingredients or may be used alone for applications in the food, pharmaceutical, nutraceutical, personal care, and industrial industries. In many embodiments the ingredients used in this invention are derived from a natural resource, and are safe to ingest. In some embodiments, the spheres are formed using a centrifugal tumbling-granulating-coating apparatus.

### CROSS-REFERENCE TO RELATED APPLICATIONS

This application claims benefit of U.S. Provisional Application Number 61/703,053, filed Sep. 19, 2012, which is hereby incorporated herein by reference in its entirety.

### TECHNICAL FIELD

The disclosure relates generally to microspheres. In many embodiments, the microspheres are suitable for use in such commercial applications as coatings. In other embodiments, the microspheres may be used in connection with pharmaceutical, nutraceutical, food, personal care and industrial applications as a carrier or as a source of fiber. Some embodiments pertain to methods for preparing and using such microspheres.

### BACKGROUND

Spherical particles, or spheres, are used in many industries. In the food industry, for example, spheres are used to decorate cakes, cupcakes, cookies or desserts. They may be sprinkled on top or added within the matrix of the product. These spheres are typically colored and can be flavored. In the food industry, spheres are also called nonpareils, sprinkles, hundreds and thousands, dragées, or pearls. They are typically made from sugar, but may be made of other ingredients, such as chocolate. Sugar spheres are typically composed of 90 to 95 percent sucrose using starch as the binder at 5 to 10 percent. They are high calorie particles that serve a decorative purpose, but, other than calories from the carbohydrate content, do not add any nutritional value to the finished product.

Spheres also are used in the pharmaceutical industry in applications such as multi-particulate solid dosage delivery systems. The spheres, generally referred to as pellets or nonpareils in the pharmaceutical industry, can act as the seed for drug layering and subsequent coating. These coated spheres then may be incorporated into tablets, capsules, powders, suspensions or other dosage forms. When used for this purpose, the surface of the spheres should be as smooth as possible to allow for a uniform layering of the drug or other active ingredient resulting in an accurate dose in the finished product. The spherical cores should also have good flowability and should provide enough mechanical strength to withstand the further processing in the coating process as well as in tableting or capsule filling.

Using spheres in controlled release solid dosage forms allows the tablet, capsule or powder to release the active ingredient over time at a controlled rate. This dosage form not only insures an accurate dose, but also distributes the dose throughout the gastrointestinal tract instead of in one location. This is believed to decrease “dose dumping” and related toxic effects from the active ingredient.

Beyond the pharmaceutical industry, spheres may be used more broadly with coatings for controlled release formulas. In the personal care industry, spheres are often used for aesthetics. They can be made from a wide variety of ingredients including waxes, sugars, sugar alcohol, alginates, microcrystalline cellulose, etc.

Currently available products include the traditional sugar spheres, and more recently, microcrystalline cellulose spheres and some starch spheres. Sugar spheres are cost-effective, but processing can present some difficulties. During the coating process, sugar spheres can become tacky and hinder drug layering. Because the sugar is water soluble and can dissolve when it comes in contact with aqueous coatings, the sugar cores can become difficult to coat and can stick to each other and to the surface of the pan.

Microcrystalline cellulose spheres (MCC) provide an inert core for drug layering and coating. They are dense spheres and most have a relatively smooth surface to allow for uniform dosage of the active ingredient being applied to the core, and are not water soluble. In many cases, however, the smaller sizes of microcrystalline cellulose spheres are not uniform in particle size distribution. Also, MCC spheres are commercially cost prohibitive for economical finished formulations in the pharmaceutical industry and especially for the food industry. They can also absorb moisture from the coating process, decreasing stability of the finished product.

Some studies have shown the dissolution of a coated sugar core may decrease over time, thus reducing the dosage of the active ingredient being released while the dissolution of a coated microcrystalline cellulose core may increase over time, increasing the amount of active ingredient being released. Sugar spheres are not uniform in particle size, and can react with active ingredients.

Copending application Ser. No. 13/623,591 (Freers et al. assignors to Grain Processing Corporation of Muscatine, Iowa), published as U.S. Publication no. 2013/0071479, discloses microspheres made in many embodiments from starch and maltodextrin. These microspheres may be manufactured by a combination of rotor granulation and powder layering in the rotor granulator. The spheres are small and spherical with a uniform particle size. The spheres are used as cores for coating an active ingredient with an additional coating of controlled release polymer and other carrying applications.

These spheres provide excellent properties for many applications. These spheres in many embodiments constitute digestible carbohydrates and nutritionally provide 4 calories per gram. In some cases it is desirable to provide spheres that are not digestible or that are digestible to a lesser extent.

### SUMMARY

Spheres may be made from bran and a binder, such as corn bran in combination with maltodextrin. The corn bran used to make the spheres described in this disclosure is not water soluble and is not digestible, so the spheres made from the corn bran are less soluble than sugar spheres. This allows for drug layering with an insoluble outer layer, and provides a fiber source for the body. They may be used as-is in a tablet or capsule to provide a fiber source, or they can be used to carry flavors, colors, sweeteners, or active ingredients.

Also contemplated is a method for preparing spheres. The spheres may be formed in a centrifugal tumbling-granulating-coating apparatus.

In some embodiments, not mutually exclusive with regard to the heretofore described embodiments, the invention contemplates coated spheres, and in other embodiments, the invention contemplates methods for preparing coated spheres.

### BRIEF DESCRIPTION OF DRAWINGS

FIGS. 1-4 are scanning electron micrographs of spheres produced in Example 1 below.

FIG. 5 is a scanning electron micrographs of a cross-section of one of the spheres shown in FIGS. 1-4.

### DETAILED DESCRIPTION

Spheres may be created from fiber and a binder. Any suitable binder may be used, but the binder is preferably a carbohydrate binder, such as an oligosaccharide binder, and preferably is a malto-oligosaccharide, such as a maltodextrin. Other binders including but not limited to, polyvinylpyrrolidone, gums, starch, hydroxypropyl methylcellulose (HPMC) may be used, as well as combinations of any of the foregoing. The fiber and maltodextrin each may be derived from corn, wheat, rice, pea, potato, or other sources.

Any suitable fiber may be used to prepare the spheres. Dietary fiber plays a critical role in the promotion of health and prevention of disease. General health concerns including obesity, heart health, and other diet issues. Consumers continue to look for healthy options in their diets; however, they cannot always get the recommended amount of dietary fiber from the foods they eat. Nutritional supplements, beverages, and nutritional bars filled with fiber can fortify the diet. Often the fiber is incorporated into the tablet, capsule, bar, etc. as a powder or a granule.

There are two basic kinds of fiber: soluble and insoluble. Soluble fiber is defined as fiber that is readily fermented in the colon into gases and physiologically active byproducts. Insoluble fiber is metabolically inert. It absorbs water throughout the digestive system, increases bulk, softens stools, eases defecation and shortens transit time through the intestinal tract. In addition, consuming fibers that add bulk to the diet may increase satiety.

One line of fiber products available commercially is TRUBRAN® corn bran, sold by Grain Processing Corporation of Muscatine, Iowa. TRUBRAN® is sold as a powder. These fibers have a clean flavor profile and light color making them easy to incorporate into a variety of products. The fibers can promote the passage of foods through the digestive system, increase bulk, and soften the stool, alleviating constipation. TRUBRAN® fibers are ideal for nutritional beverages, nutritional bars, and supplements such as soft chews, tablets, capsules, and powders to achieve an excellent source of fiber.

Similarly, any suitable binder may be employed. The binder is preferably a carbohydrate and more preferably an oligosaccharide, by which is contemplated any species comprised of plural saccharide units, whether linked by 1-4 linkages, 1-6 linkages, or otherwise. For example, malto-oligosaccharides and mixtures thereof, as well as other oligosaccharides, may be employed as binders. By “malto-oligosaccharides” is contemplated any species comprising two or more saccharide units linked predominately via 1-4 linkages, and including maltodextrins and syrup solids. In preferred embodiments, at least 50 percent of the saccharide units in the malto-oligosaccharide are linked via 1-4 linkages. More preferably, at least about 60 percent of the saccharide units are linked via 1-4 linkages; even more preferably, at least about 80 percent of the saccharide units are so linked. The malto-oligosaccharides may include saccharide species having an odd DP value, and the profile may be partially defined by a saccharide species having a DP value of 1, for example, dextrose or sorbitol. Reduced malto-oligosaccharides may be employed as binders.

While the invention finds applicability with respect to any malto-oligosaccharide mixture, the invention is particularly applicable to malto-oligosaccharide species in which at least a portion of the malto-oligosaccharides in the mixture have a DP value greater than 5. Preferably, at least one of the malto-oligosaccharide species in the mixture has a DP value of 8 or more. More preferably, at least one species has a DP value of at least 10. For example, in preferred embodiments of the invention, at least 80 percent of the malto-oligosaccharide species in the mixture have a DP greater than 5, and at least 60 percent may have a DP greater than 8. In another embodiment, at least 80 percent of the malto-oligosaccharides species have a DP greater than 10. In some embodiments of the invention, the DP profile of the starting mixture is such that at least 75 percent of the malto-oligosaccharides species in the mixture have a DP greater than 5 and at least 40 percent of the species in the mixture have a DP greater than 10. Such starting materials may be obtained conventionally, for example, by the partial hydrolysis of starch. Further teachings concerning maltodextrins and malto-oligosaccharides more generally can be found in U.S. Pat. Nos. 7,728,125 (“Reduced malto-oligosaccharides”); 7,595,393 (“Reduced malto-oligosaccharides”); 7,405,293 (“Reduced malto-oligosaccharides”); 7,091,335 (“Derivatized reduced malto-oligosaccharides”); 6,919,446 (“Reduced malto-oligosaccharides”); and 6,613,898 (“Reduced malto-oligosaccharides”), all assigned to Grain Processing Corporation of Muscatine, Iowa.

Suitable malto-oligosaccharides are sold as maltodextrins under the trademark MALTRIN® by Grain Processing Corporation of Muscatine, Iowa. The MALTRIN® maltodextrins are malto-oligosaccharide products, each product having a known typical DP profile. MALTRIN® maltodextrins suitable as binders include, for example, MALTRIN® M040, MALTRIN® M100, MALTRIN® M150, and MALTRIN® M180. The low dextrose equivalence of maltodextrin is believed to enhance the stability of the spheres when active ingredients are applied to the surface.

The fiber and maltodextrin may be present in any suitable amounts relative to one another. Preferably, the fiber is present in an amount of at least 50%, more preferably, at least 55%, more preferably, at least 60%, more preferably, at least 65%, more preferably, at least 70%, more preferably, at least 75%, and more preferably, at least 80%, by dry weight of the total of fiber and maltodextrin. In one form, stronger binders may be used at lower levels such that the percent of fiber may be increased relative to the binder. Further, other components may be included in the microspheres.

Any suitable process may be employed to prepare the spheres. In one process, spheres are produced by rotor granulation, using a centrifugal tumbling-granulating-coating apparatus, such as the GRANUREX® GXR Rotor Granulator (commercially available from Freund-Vector Corporation). In this process, the ingredients are introduced into the rotor granulating apparatus and granulated to produce spheres. In many embodiments, this process is followed by a powder layering step, in which the sphere created by rotor granulation is used as the seed for the powder-layering process and larger spheres are created. The powder layering process may be begun in the same rotor granulation bowl when the rotor granulation is concluded. Alternatively, if desired, the spheres can be screened before the powder layering step to select a narrow range of particle size, and subsequently built up to much larger, more uniform spheres using a powder layering process. The powder layering process may be repeated if desired.

Spheres produced by rotor granulation can be made not to agglomerate, with high processing efficiencies resulting in excellent process yields. Other granulation, extrusion, or spheronization techniques may be used to produce these fiber spheres.

The spheres thus prepared are roughly spherical. The shape, diameter and size are preferably uniform and the surface is preferably as smooth as economically feasible. Surface smoothness is advantageous if the spheres are used for layering an active ingredient to attain a more uniform dosage. The spheres in many embodiments do not have the same reactive properties that sugar spheres have, so they improve the stability of the finished dosage form as compared with sugar spheres. The spheres can be made to have low friability and good flow properties. They are also a good source of fiber for the diet. In some embodiments it is believed that the spheres allow for sustained release of fiber in the stomach and intestine.

The spheres may be formed in various sizes and can be made to have a small uniform particle size with narrow particle size distribution. The methods described herein can be used to provide substantially uniform microspheres. As used herein, the term “substantially uniform” means that the microspheres produced according to the methods described herein have a narrow particle size distribution and have a high sphericity without the need for screening to adjust particle size (e.g., by fluidized bed separation or screen filtering), although such techniques can be used if desired. Sphericity (Ψ) is a measure of the roundness of an object. Sphericity is the ratio of the surface area of a sphere (which has the same volume as the particle being compared) to the surface of the particle being tested. Sphericity can be calculated according to the following formula:

$$Ψ = \frac{{{π}^{\frac{1}{3}} ( 6 {V}_{p} )}^{\frac{2}{3}}}{{A}_{p}} ,$$

where Vp is the volume of the sphere and Ap is the surface area of the sphere. By some approaches, the spheres may have a sphericity value of at least about 0.6, in another aspect at least about 0.7, in another aspect at least about 0.8, and in yet another aspect at least about 0.9. Sphericity may be determined by aspect ratio using a Sympatec, Inc. GMBH QICPIC with RODOS/L Dry Dispersing Module. The population of spheres produced may include some microspheres having a lower sphericity value while providing the desired high sphericity value for the overall population of spheres.

When initially formed, the initial spheres can have, for example, a size of 100 to 200 microns in size. In other forms, the initial spheres have a mean particle size of about 100 microns, 110 microns, 120 microns, 130 microns, 140 microns, 150 microns, 160 microns, 170 microns, 180 microns, 190 microns, and 200 microns and up. This can be the final product if desired. These spheres can then be built to a larger desired size by using a powder layering process. Spheres as large as 2 millimeters can be prepared using such a process. Generally, coating the spherical cores will increase the sphericity value. In one aspect, a population of coated spheres has a mean sphericity value of at least about 0.6, in another aspect at least about 0.7, in another aspect at least about 0.8, and in yet another aspect at least about 0.9.

The spheres can also be used to transport and/or deliver materials such as pharmaceuticals. For example, the spheres can be used to deliver pharmaceuticals after the spheres are ingested. In one form, the spheres can be coated with pharmaceuticals such as ibuprofen and the like. The spheres may optionally be incorporated into a capsule or other form for ingestion.

The following Examples are intended to illustrate an embodiment of the present invention, but should not be construed more generally as limiting the invention in scope.

### EXAMPLE 1

This Example illustrates the preparation of 70% fiber spheres using the combination rotor granulation and powder layering process.

Fiber spheres were manufactured on a GRANUREX® GXR Rotor Granulator equipped with an ATU Mini air atomized spray system. The process used a two-phase approach, with the first step creating small fiber cores and the second phase powder layering the fiber onto the cores to create larger, uniform spheres.

The Granurex® bowl was initially charged with a powder blend of 70% TRUBRAN® F75M Corn Bran and 30% MALTRIN® M100 Maltodextrin. Both ingredients are fine particle size products manufactured by Grain Processing Corporation. Using water as the granulating medium, the powder blend was spherically granulated and very small and uniform spheres were manufactured. Phase II involved using these spheres as seeds for the powder layering process to create larger uniform spheres. The spherical cores were removed from the bowl and a screen cut was taken through a US Standard 25 mesh screen and on a US Standard 70 mesh screen to capture the spheres between 200 and 300 microns. These spheres were returned to the GXR bowl. Using the precision powder feeder and powder delivery system on the GXR, TRUBRAN® F75M Corn Bran was layered onto the seeds. A 30% MALTRIN® M100 Maltodextrin in water solution was used to adhere the fiber onto the spheres to approximately double their size. Again the spheres were screened to select the product remaining on a US Standard 50 mesh screen and the above 300 micron spheres were returned to the GXR bowl. The 30% MALTRIN® M100 Maltodextrin in water solution was used to continue to bind fiber onto the spheres increasing their size while maintaining a very uniform, spherical shape. A third screen cut was made to retain the spheres above a US Standard 35 mesh screen (500 microns) and the spheres were returned to the GXR bowl and increased in size to 1000 microns using the 30% MALTRIN® M100 Maltodextrin in water solution and TRUBRAN® F75M Corn Bran.

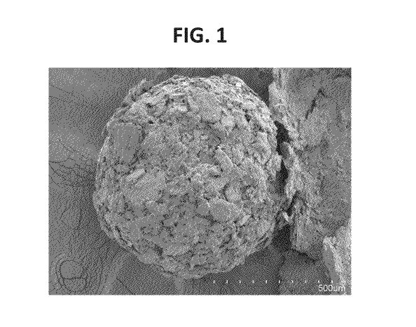

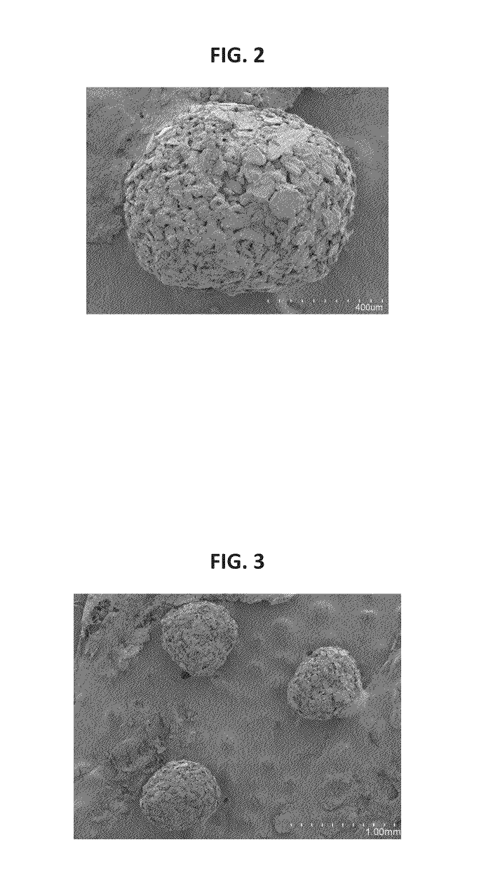

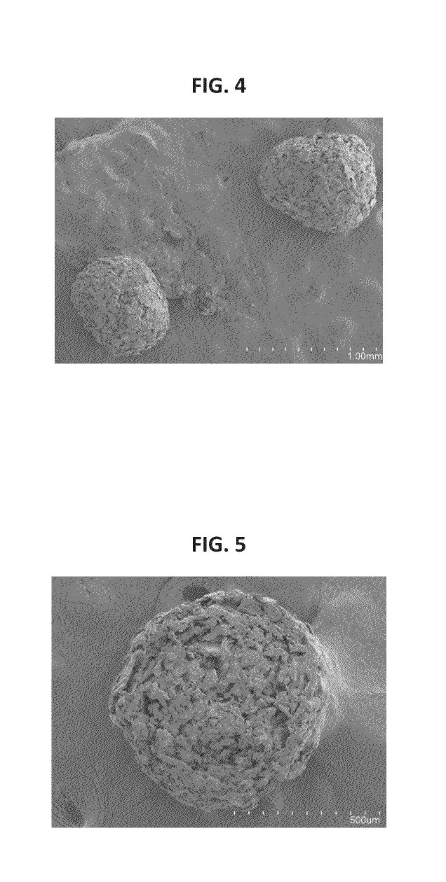

The resulting spheres were extremely uniform in size and had a very smooth surface. They exhibited excellent flow properties. They had a bland flavor. Exemplary spheres are shown in FIGS. 1-4, with a cross-sectional view of one of the spheres shown in FIG. 5.

### EXAMPLE 2

The spheres produced in accordance with Example 1 are coated with ibuprofen and are dosed into capsules.

The spheres may be used in a variety of products and applications. For example, the spheres may also be used in nutritional applications to provide a source of fiber, such as in granola bars, high fiber/protein bars, cereals and the like. In some embodiments, a method for providing nutrition to an animal is provided. The animal may be, for example, a human, or may be swine, bovine, or other animal. The method comprises ingesting or making available for ingestion an amount of the fiber spheres suitable for providing nutrition to the animal. The fiber spheres may be provided in a food product and may comprise any desired proportion of that food product, for example, 10%, 20%, 30%, 40%, 50%, 60%, 70%, 80%, or 90% of the food product, or the fiber spheres may be ingested as is.

It is thus seen that spheres can be prepared from fiber and a binder. They may be used for their functional properties or they be used because they can deliver a source of fiber for the diet. Also, spheres can be made to have an economical advantage over microcrystalline cellulose spheres, and to have a dietary fiber and stability advantage over traditional sugar spheres.

All references cited herein are hereby incorporated by reference in their entireties.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A microsphere composition comprising a plurality of microspheres each comprising a spherical core comprising fiber and a binder, the fiber being present in an amount of at least about 50% by dry weight of the total fiber and binder.

2. The microsphere composition of claim 1, wherein the fiber is an insoluble fiber.

3. The microsphere composition of claim 2, wherein the fiber is corn bran.

4. The microsphere composition of claim 1, wherein the binder is an oligosaccharide.

5. The microsphere composition of claim 4, wherein the binder is a malto-oligosaccharide.

6. The microsphere composition of claim 5, wherein the binder is maltodextrin.

7. The microsphere composition of claim 1, wherein the fiber is corn bran and the binder is maltodextrin and the corn bran is present in an amount of at least about 60% by dry weight of the total corn bran and maltodextrin.

8. The microsphere composition of claim 7, wherein the corn bran is present in an amount of at least about 70% by dry weight of the total corn bran and maltodextrin.

9. The microsphere composition of claim 1, wherein the microspheres have a mean sphericity of at least about 0.6.

10. The microsphere composition of claim 1, wherein the microspheres have a mean sphericity of at least about 0.9.

11. The microsphere composition of claim 1, wherein the microspheres have a mean particle size of at least about 100 microns.

12. The microsphere composition of claim 1, further comprising a coating on the spherical cores.

13. A process for preparing microspheres, the process comprising granulating a powder mixture of fiber and a binder using an aqueous liquid as a granulating medium to provide microspheres each having a spherical core comprising fiber and a binder, the fiber being present in an amount of at least about 50% by dry weight of the total fiber and binder.

14. The process of claim 13, further comprising applying a coating to the spherical cores using a powder layering process.

15. The process of claim 13, wherein the fiber is corn bran and the binder is maltodextrin and the corn bran is present in an amount of at least about 60% by dry weight of the total corn bran and maltodextrin.

16. The process of claim 15, wherein the corn bran is present in an amount of at least about 70% by dry weight of the total corn bran and maltodextrin.

17. (canceled)

18. The process of claim 13, wherein the microspheres have a mean sphericity of at least about 0.9.

19. The process of claim 13, wherein the microspheres have a mean particle size of at least about 100 microns.

20. A method of providing fiber to an animal, the method comprising the step of ingesting or making available for ingestion an amount of microspheres suitable for providing nutrition to the animal, the microspheres each comprising a spherical core comprising fiber and a binder, the fiber being present in an amount of at least about 50% by dry weight of the total fiber and binder.

21. A microsphere composition comprising a plurality of microspheres each comprising a spherical core and a pharmaceutical coating, the spherical core comprising fiber and a binder, the fiber being present in an amount of at least about 50% by dry weight of the total fiber and binder.

22-31. (canceled)
